(19) United States
(12) Patent Application Publication
(10) Pub. No.: US 2010/0198185 A1
(43) Pub. Date: Aug. 5, 2010

(54) MULTI-CHAMBER BAG FOR USE WITH ENTERAL NUTRITION

(76) Inventor: Irmgard Görisch, Leonding (AT)

Correspondence Address: HAMMER & ASSOCIATES, P.C., 3125 SPRINGBANK LANE, SUITE G, CHARLOTTE, NC, 28226, US

(21) Appl. No.: 12/665,359

(22) PCT Filed: Jun. 19, 2008
(86) PCT No.: PCT/EP08/04911

§ 371 Date: Apr. 20, 2010

(30) Foreign Application Priority Data
Jun. 21, 2007 (DE) 10 2007 028 733.1

(51) Int. Cl. A61B 19/00

(52) U.S. Cl. 604/410

(57) ABSTRACT

The container according to the invention for use in enteral nutrition is characterized in that the container is a multi-chamber bag with at least two chambers separated from one another, wherein at least one chamber contains an enteral nutritive solution with a first energy content and at least one chamber contains a fluid for covering the fluid requirement with an energy content which is less than the energy content of the enteral nutritive solution. Since, with the multi-chamber bag according to the invention, both the enteral nutritive solution and the fluid also required for covering the fluid requirement are made available directly, the application in the case of enteral nutrition is simplified.

The invention relates to a multi-chamber bag for use in enteral nutrition, which contains an enteral nutritive solution.

In medicine, a distinction is made between enteral and parenteral nutrition. Parenteral nutrition differs from enteral nutrition by the fact that it takes place by bypassing the gastrointestinal tract. Enteral and parenteral nutritive solutions therefore also differ fundamentally in composition.

Enteral nutritive solutions must meet requirements with regard to the energy and nutritive content. They generally contain proteins, carbohydrates, fats, vitamins and mineral substances. Enteral nutritive solutions may also contain ballast substances.

Industrially produced enteral nutritive solutions are made available as probe nutrition in rigid or flexible containers. Bottles or bags are used as containers. The containers comprise a connection piece for the connection of a transfer system, with which the enteral nutritive solution is transferred from the container into the patient's stomach. The transfer of the nutritive solution can take place by gravity or by means of a pump.

The known transfer systems comprise a tube line, which at one end comprises a connector for the connection to the connection piece of the nutritive solution container and at the other end a connector for the connection to the connection piece of a probe tube. When the nutritive solution is conveyed by means of a pump, a section of the tube line is designed as a pump segment which is placed into a roller pump.

Whereas all the nutritional constituents can be prepared in just one single solution in the case of enteral nutrition, the nutritional constituents, in particular protein sources, carbohydrates and fats, have to be preserved separately in the case of parenteral nutrition for reasons of stability.

EP 0 790 051 B1 describes a bag for use in parenteral nutrition, which contains a plurality of chambers which are separated from one another for the separate storage of the individual nutritional constituents. A fluid connection is produced between the individual chambers only shortly before administration of the parenteral nutritive solution, so that the various constituents can be mixed together.

Connection pieces designed as break-off parts, which are welded into the weld seams of the bag, are used to produce the fluid connection. Alternatively, the weld seams can also be designed as separable or tearable seams. These seams are referred to us peel seams.

Multi-chamber bags for use in parenteral nutrition are also known from EP 0 619 998 B1, EP 0 893 982 B1 and EP 0 883 396 B1.

Since stability problems due to the mixing of the constituents, which include protein sources, carbohydrates and fats, do not in principle arise with the storage of enteral nutritive solutions, no containers are known for use in enteral nutrition that comprise a plurality of compartments for accommodating the individual constituents of the enteral nutritive solution.

The patient's fluid requirement cannot be covered with the industrially produced enteral nutritive solutions alone. It is therefore necessary also to administer a fluid to the patient apart from the enteral nutritive solution. This fluid can have a lower energy content. In the simplest case, water, which has no nutritional value, is administered to the patient apart from the enteral nutritive solution.

For the administration of fluids for covering the fluid requirement, use is made in the prior art of separate containers which are also referred to as hydrobags.

There is known from EP 1 795 169 A1 a container which is divided by a partition wall into two chambers of equal size. A common outlet opening, which is connected to both chambers, is used to empty the chambers. Connected to the outlet opening is a shut-off or mixing device, with which the fluids contained in the chambers can be removed in the desired dosage or one after the other. One chamber is intended to accommodate an enteral nutritive solution and the other chamber tea or water. The two chambers should be able to be topped up with enteral nutritive solution or tea or water. For this reason, the chambers each comprise a filling opening, which can be closed with a cap. The enteral nutritive solution, which is packaged protected against light and oxygen in tubular bags made of aluminium composite foil, is not supposed to be put into the container until just before administration.

The problem underlying the invention is to simplify enteral nutrition with a sufficient fluid supply.

According to the invention, this problem is solved with the features of claim 1. Advantageous embodiments of the invention are the subject-matter of the sub-claims.

The container according to the invention for use in enteral nutrition is characterised in that the container is a multi-chamber bag with at least two chambers separated from one another, wherein at least one chamber contains an enteral nutritive solution with a first energy content and at least one chamber contains a fluid for covering the fluid requirement with an energy content which is less than the energy content of the enteral nutritive solution. Since, with the multi-chamber bag according to the invention, both the enteral nutritive solution and the fluid also required for covering the fluid requirement are made available directly, the application in the case of enteral nutrition is simplified. Transfer of fluids from other containers is not necessary. Both fluids are made available together in one bag which can easily collapse when emptied and can be produced cost-effectively. Since the bag can collapse, ventilation of the bag is in principle not required. As a result, there is also no increased contamination risk.

A decisive advantage of the multi-chamber bag according to the invention lies in the fact that both the enteral nutritive solution and the fluid for covering the fluid requirement are made available in such a way that account can be taken of the individual needs of the patient with regard to the composition and quantity of the two fluids.

For example, either the enteral nutritive solution alone can be administered to the patient from one chamber, or both the enteral nutritive solution and the fluid for covering the fluid requirement can be administered. Both fluids are available in the multi-chamber bag. If the multi-chamber bag comprises more than two chambers, different quantities of fluids can be made available which can be administered depending on the individual needs of the patient. For example, a plurality of chambers can be provided for enteral nutritive solutions and/or a plurality of chambers can be provided for fluids for covering the fluid requirement, said chambers having different volumes and accommodating different quantities of fluid. Just individual ones or all of the chambers are then emptied depending on the patient's individual needs.

In order to cover the patient's energy requirement, the enteral nutritive solution has an energy content of at least 0.5 kcal/ml, preferably at least 0.8 kcal/ml, particularly preferably at least 1 kcal/ml. It preferably contains an albumen source, carbohydrates and fats. Milk and soya proteins can be used as protein sources. Polysaccharides and oligosaccharides can be used as carbohydrates. As sources of fats, consideration can be given for example to soya, sunflower oils, coconut and milk fats as well as fish oil, MCT (medium chain triglyceride), palm kernel oil, rape-seed oil and structured triglycerides. Vitamins, trace elements and electrolytes such as ballast substances can also be added to the enteral nutritive solution.

The fluid for covering the fluid requirement differs from the enteral nutritive solution essentially by the lower energy content. In the simplest case, the fluid requirement is covered by water.

The at least one chamber that is filled with the enteral nutritive solution preferably has a larger volume than the at least one chamber that is filled with the fluid for covering the fluid requirement. The one chamber preferably contains 750 to 1500 ml of enteral nutritive solution, whilst the other chamber contains 250 to 750 ml of fluid.

In a preferred embodiment, the multi-chamber bag comprises means for producing a fluid connection between the individual chambers, which contain enteral nutritive solution or fluid for covering the fluid requirement. In this embodiment, a single connection piece suffices both for the removal of the enteral nutritive solution as well as the fluid for covering the fluid requirement. After administration of the enteral nutritive solution, the fluid connection can be produced between the chambers and, instead of the already administered nutritive solution, the fluid for covering the fluid requirement can be administered. In principle, however, the two fluids can be mixed in advance.

The administration of the nutritive solution or the fluid for covering the fluid requirement can take place by gravity or by means of a metering pump. For this purpose, the multi-chamber bag preferably comprises a hanger on which the container can be suspended on a stand or suchlike. This hanger is arranged on the side of the multi-chamber bag lying opposite the connection piece for the removal of the fluids.

An alternative embodiment makes provision such that not only the at least one chamber that is filled with the enteral nutritive solution, but also the at least one chamber that is filled with the solution for covering the fluid requirement comprises a connection piece for the removal of fluid.

With this embodiment, each chamber can be emptied via the respective connection piece, a fluid connection between the chambers not having to be produced.

In the alternative embodiment, the multi-chamber bag preferably also comprises a hanger on the side lying opposite the connection piece for removing the fluid for covering the fluid requirement. In order to remove the enteral nutritive solution or the fluid for covering the fluid requirement, the multi-chamber bag merely has to be turned round and suspended on the respective hanger.

A further particularly preferred embodiment makes provision such that the multi-chamber bag is made available together with a device for transferring the enteral nutritive solution and/or the fluid for covering the fluid requirement to the patient. The multi-chamber bag and the transfer system are preferably packaged in a sterile manner in a packaging. As transfer systems, use may be made of all known transfer devices that comprise a tube line, which at one end comprises a connection piece for the multi-chamber bag and at the other end a connection piece for a nutritional probe. The packaging enclosing the multi-chamber bag and the transfer system is again preferably a bag.

The multi-chamber bag can be produced from materials known to the person skilled in the art according to the known methods.

In the multi-chamber bag, the means for producing a fluid connection between the chambers is preferably designed as separable or tearable seams, so-called peel seams. In the alternative embodiments, in which a fluid connection between the chambers is not to be produced, the seams which separate the individual chambers from each other are not separable seams.

Various examples of embodiment of the invention are explained in greater detail below by reference to the drawings.

In the figures:

FIG. 1 shows a simplified schematic representation of a first example of embodiment of the multi-chamber bag according to the invention for use in enteral nutrition, which comprises two chambers,

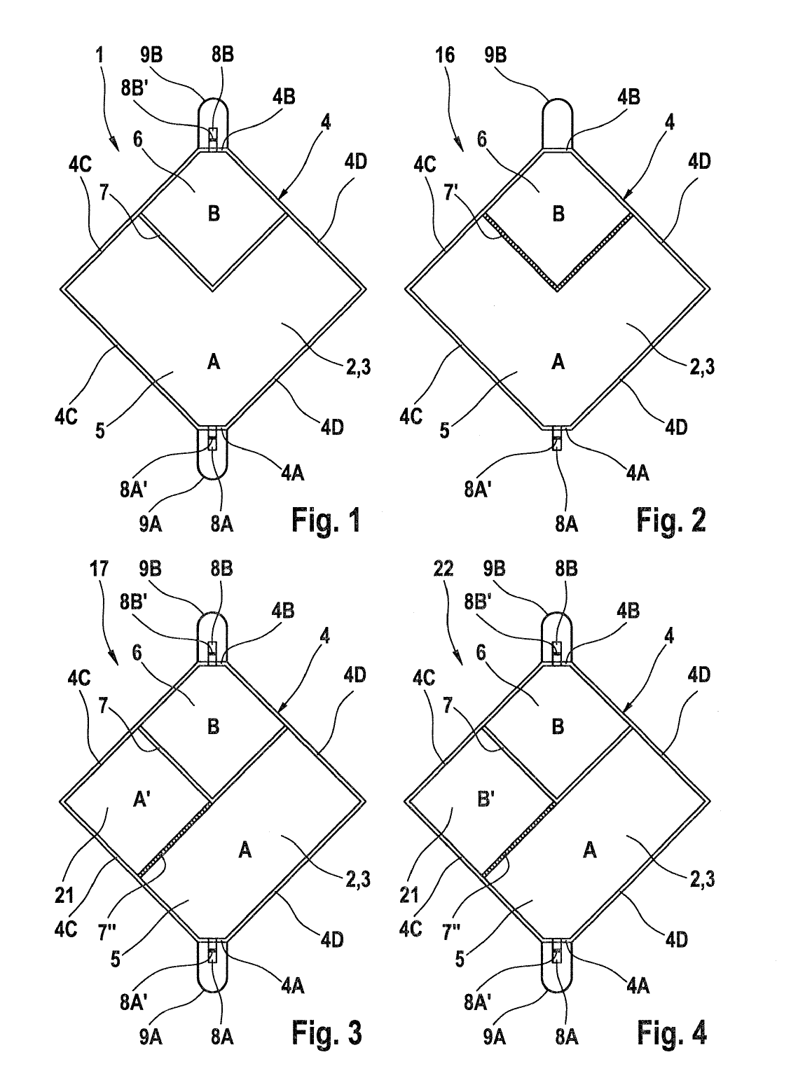

FIG. 2 shows a second example of embodiment of the multi-chamber bag according to the invention in a schematic representation, which comprises two chambers,

FIG. 3 shows a further example of embodiment of the multi-chamber bag according to the invention, which comprises three chambers,

FIG. 4 shows an alternative example of embodiment of the multi-chamber bag according to the invention with three chambers and

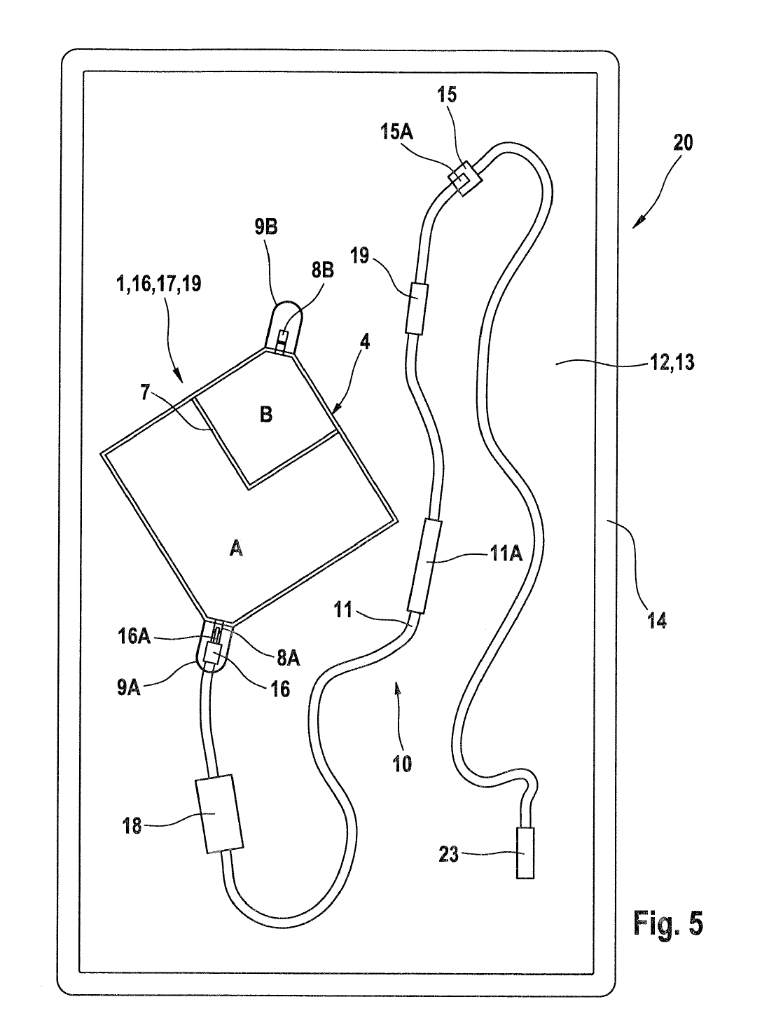

FIG. 5 shows an arrangement in a simplified schematic representation, which comprises the multi-chamber bag according to the invention together with a transfer system, the multi-chamber bag and the transfer system being enclosed by a packaging.

FIG. 1 shows in a simplified schematic representation an example of embodiment of the multi-chamber bag according to the invention for use in enteral nutrition. The multi-chamber bag according to the invention is a polymer bag in the present example of embodiment.

In the present example of embodiment, bag 1 comprises two flexible films 2, 3 lying one above the other, which are welded at their edges with a peripheral weld seam 4, which in the position shown in FIG. 1 comprises a lower section 4A and an upper section 4B as well as two side sections 4C and 4D. The bag can however also comprise a film tube, which is welded at the upper and lower side.

The bag is divided into a lower chamber 5 and an upper chamber 6. Lower chamber 5 has a larger volume than upper chamber 6. For example, the lower chamber can accommodate 1500 ml of fluid, whereas the upper chamber can accommodate 500 ml of fluid. Both chambers 5, 6 are separated from one another by a non-separable weld seam 7, which runs in the upper third of the bag between lateral sections 4C, 4D of peripheral weld seam 4.

Bag 1 comprises two connection pieces 8A and 8B, whereof one connection piece 8A is welded into lower section 4A of peripheral weld seam 4 and other connection piece 8B is welded into upper section 4B of peripheral weld seam 4.

Connected to connection pieces 8A and 8B is a transfer system, which will be described in detail by reference to FIG. 5. A connection piece for the connection of a transfer system to a bag for accommodating an enteral nutritive solution is known for example from EP 1 010 412 A2 or EP 0 830 874 B1, to which reference is expressly made for the purpose of disclosure. Such a connection piece is also referred to as a port.

Connection pieces 8A and 8B each comprise a pierceable membrane 8A′ and 8B′, which are represented solely in outline. The pierceable membranes seal both chambers 5, 6 in a sterile manner.

The bag comprises a lower hanger 9A in the region of lower section 4A of peripheral seam 4 and an upper hanger 9B in the region of upper section 4B of peripheral seam 4. Both hangers 9A, 9B are designed as straps, which are moulded to the lower and upper section of the bag. As hangers, use may however also be made of cutouts in the upper and lower sections of the bag or its weld seams.

Lower chamber 5 with the larger volume is filled with an enteral nutritive solution A which contains various constituents, including a protein source, carbohydrates and fats. Enteral nutritive solution A alone cannot cover the patient's fluid requirement. In order to balance the fluids, therefore, the patient requires a further fluid B which is contained in upper chamber 6. In the present example of embodiment, this fluid is water (H₂O).

Enteral nutritive solution A differs from fluid B for covering the patient's fluid requirement by the fact that the enteral nutritive solution has a much higher energy content than fluid B for covering the fluid requirement. The enteral nutritive solution has an energy content of at least 0.5 kcal/ml, preferably at least 0.8 kcal/ml, particularly preferably at least 1 kcal/ml, whereas the fluid for covering the fluid requirement has an energy content which is less than 0.5 kcal/ml, and is preferably water which has no energy content.

A transfer system 10, which is described below by reference to FIG. 5, is used for the application of enteral nutritive solution A or fluid B for balancing the fluids.

FIG. 5 shows an arrangement which comprises bag 1 and transfer system 10, wherein the bag and the transfer system are packaged in a packaging 20 in a sterile manner. Packaging 20 is in turn a bag comprising films 12, 13 lying one above the other, which are welded together with a peripheral weld seam 14. Bag 20 can however also comprise a film tube which is welded at the upper and lower side. At least a part of peripheral weld seam 14 is designed as a separable or tearable weld seam (peel seam). It is therefore possible to tear open packaging 20 in a straightforward manner in order to remove bag 1 and transfer system 10.

Transfer system 10 comprises a tube line 11, a connector 16 for the connection of the transfer system to one of the two connection pieces 8A, 8B of bag 1 being provided at one end of tube line 11 and a connector 23 for the connection of the transfer system to a corresponding connector of a probe tube (not shown) for the enteral nutrition being connected to the other end of the tube line. The conveying of the fluids can take place by gravity alone. A metering pump can however also be used. The known metering pumps are peristaltic hose pumps, into which a tube line section 11A of tube line 11 is inserted.

A drip chamber 18 and a tube clamp 19 are located in tube line 11. Before use, the tube line is sealed tight with a closure part 15. Closure part 15 contains a break-off part 15A, which is broken off to open the tube line, so that a fluid connection is produced.

In packaging 20, connector 16 of the transfer system is already connected to lower connection piece 8A of bag 1. The transfer system can however also simply be placed in the bag. Connector 16 of the transfer system comprises a spike 16A, with which membrane 8A′ or 8B′ of connection pieces 8A or 8B can be pierced, so that a fluid connection can be produced to both chambers 5, 6 of bag 1.

Enteral nutrition and fluid balancing can take place in a straightforward manner with the system according to the invention. For this purpose, enteral nutritive solution A is fed from lower chamber 5 under the effect of gravity or by means of a metering pump via tube line 11 of transfer system 10 and the probe tube (not shown) to the patient. The bag is suspended on upper hanger 9B. In order to cover the fluid requirement, the bag is turned round and suspended on lower hanger 9A, connector 16 of transfer system 10 being connected to the then lower connection piece 8B, so that the then lower chamber 6 filled with water B is emptied.

The two chambers can be emptied at different times without a connection existing between the chambers. If the administration of further fluid is not required, the chamber filled with water does not need to be emptied.

Chamber 5 filled with enteral nutritive solution A basically has a larger volume than chamber 6 filled with water B. It is however also possible to fill the larger chamber with water and the smaller chamber with enteral nutritive solution.

FIG. 2 shows an alternative example of embodiment of the two-chamber bag according to the invention. Two-chamber bag 16 of FIG. 2 differs from bag 1 of FIG. 1 in that weld seam 7, which separates two chambers 5, 6 from one another, is not a permanent seam, but at least in a sub-section is formed as a peel seam 7′ which can be separated or torn open manually. Furthermore, bag 12 comprises only lower connection piece 8A and upper hanger 9B, as viewed in the position shown in FIG. 2, but not an upper connection piece and a lower hanger. Otherwise, bags 1 and 16 do not differ from one another. The same reference numbers are therefore also used for the parts corresponding to one another.

Bag 12 is suspended on hanger 9B for application of the enteral nutritive solution, so that enteral nutritive solution A can be conveyed from chamber 5 under the effect of gravity or by means of a metering pump. Peel seam 7′ is severed manually in order to administer fluid B to cover the fluid requirement, so that fluid B, water for example, can be administered to the patient. In this example of embodiment, it is not necessary to turn the bag round and to reconnect the connector of the transfer system.

FIG. 3 shows a further example of embodiment, which differs from the examples of embodiment described by reference to FIGS. 1 and 2 in that bag 17 comprises three chambers. The parts corresponding to one another are again provided with the same reference numbers.

The example of embodiment of FIG. 3 differs from the example of embodiment of FIG. 1 also by a further weld seam 7″, which runs from permanent seam 7 to side section 4C of peripheral weld seam 4. Permanent weld seam 7 and peel seam 7″ divide bag 17 into three chambers 5, 6 and 21. Larger chamber 5 is filled with 1000 ml of enteral nutritive solution A and smaller chamber 21 with 500 ml of enteral nutritive solution A′, whilst smaller chamber 6 is filled with 500 ml of fluid B for covering the fluid requirement, especially water.

Three-chamber bag 17 of FIG. 3 permits a more precise metering of the patient's nutritional or fluid requirement than two-chamber bag 1, 16 of FIG. 1 and FIG. 2. Depending on the patient's requirement, it is possible to administer either just enteral nutritive solution A contained in chamber 5 or both nutritive solutions A and A′ contained in chamber 5 as well as in chamber 21. For this purpose, it is merely necessary to sever peel seam 7″ manually. Moreover, after turning the bag round and connection of the transfer system to the other connection piece, fluid B, in particular water, can be administered to cover the fluid requirement.

FIG. 4 shows a further example of embodiment of bag 22, which differs from the example of embodiment of FIG. 3 solely in that smaller chamber 21 is filled not with enteral nutritive solution, but with a fluid B′ for covering the fluid requirement, in particular water. Bag 22 thus permits an application of different quantities of water.

Bags 17 and 22 shown in FIG. 3 and FIG. 4 each comprise two connection pieces and two hangers. It is however also possible, in a similar manner to the example of embodiment of FIG. 2, for the bags of FIG. 3 and FIG. 4 to comprise only one connection piece and one hanger. In this case, permanent seam 7 is designed as a separable or tearable peel seam. It is however also possible to design peel seam 7″ as a permanent seam, the bag then comprising a further connection piece and a further hanger.

Although the present examples of embodiment show only two-chamber and three-chamber bags, the bag according to the invention can also comprise further chambers, which are separated from one another by permanent seams or peel seams and are filled either with an enteral nutritive solution or a fluid for fluid balancing for the patient, so that a particularly precise adaptation of the nutritional or fluid requirement to the patient's individual needs is possible. It is also possible for the chamber or chambers containing enteral nutritive solution to contain enteral nutritive solution with different compositions. Furthermore, it is possible for the chamber or chambers containing the fluid for covering the fluid requirement to contain different fluids for the fluid balancing, for example water on the one hand and for example fruit juice on the other hand. Here too, however, it is assumed that the energy content of the fluids for the fluid balancing is smaller than the energy content of the enteral nutritive solutions.

1. A multi-chamber bag for use in enteral nutrition, which contains an enteral nutritive solution, characterised in that the multi-chamber bag (1, 16, 17, 22) comprises at least two chambers (5, 6) separated from one another, wherein at least a first chamber (5) of said at least two chambers contains an enteral nutritive solution (A) with a first energy content and at least a second chamber (6) of said at least two chambers contains a fluid (B) for covering the fluid requirement with an energy content which is less than said first energy content of said enteral nutritive solution.

2. The multi-chamber bag according to claim 1, characterised in that said enteral nutritive solution (A) having an energy content of at least 0.5 kcal/ml.

3. The multi-chamber bag according to claim 1, characterised in that said enteral nutritive solution (A) containing at least two constituents selected from the group consisting of: a protein source, carbohydrates and fats.

4. The multi-chamber bag according to claim 3, characterised in that said enteral nutritive solution (A) containing a protein source, carbohydrates and fats.

5. The multi-chamber bag according to claim 1, characterised in that said fluid for covering the fluid requirement having an energy content which is less than 0.5 kcal/ml.

6. The multi-chamber bag according to claim 5, characterised in that said fluid (B) for covering the fluid requirement being water.

7. The multi-chamber bag according to claim 1, characterised in that the volume of said at least one chamber (5) which is filled with said enteral nutritive solution (A) being larger than the volume of said at least one chamber (6) which is filled with said fluid (B) for covering the fluid requirement.

8. The multi-chamber bag according to claim 1, characterised in that said at least one chamber (5), which is filled with said enteral nutritive solution (A), contains 750 to 1500 ml of enteral nutritive solution and said at least one chamber (6), which is filled with said fluid (B) for covering the fluid requirement, contains 250 to 750 ml of solution for covering the fluid requirement.

9. The multi-chamber bag according to claim 1, characterised in that means (7′, 7″) can be produced for producing a fluid connection between said at least one chamber (5) which is filled with said enteral nutritive solution (A) and said at least one chamber (6) which is filled with said fluid (B) for covering the fluid requirement.

10. The multi-chamber bag according to claim 1, characterised in that said at least one chamber (5) which is filled with said enteral nutritive solution (A) comprises a connection piece (8A) for removing said enteral nutritive solution.

11. The multi-chamber bag according to claim 10, characterised in that the multi-chamber bag (1) comprises a hanger (9B) at the side lying opposite said connection piece (8A) for removing said enteral nutritive solution (A).

12. The multi-chamber bag according to claim 1, characterised in that said at least one chamber (6), which is filled with said fluid (B) for covering the fluid requirement, comprising a connection piece (8B) for removing fluid.

13. The multi-chamber bag according to claim 12, characterised in that said multi-chamber bag (1) comprising a hanger (9A) at the side lying opposite said connection piece (8B) for removing said fluid (B) covering the fluid requirement.

14. The multi-chamber bag according to claim 13, characterised in that said at least one chamber (5) which is filled with said enteral nutritive solution (A) being separated by a separable seam (7′) from said at least one chamber (6) which is filled with said fluid for covering the fluid requirement.

15. The multi-chamber bag according to claim 13, characterised in that said at least one chamber (5) which is filled with said enteral nutritive solution (A) being separated by a non-separable permanent seam (7) from said at least one chamber (6) which is filled with said fluid (B) for covering the fluid requirement.

16. An arrangement with a multi-chamber bag according to claim 1, characterised in that said multi-chamber bag (1, 16, 17, 22) being enclosed by a packaging (20), which contains a device (10) for transferring said enteral nutritive solution (A) and/or said fluid (B) for covering the fluid requirement to the patient.

17. The arrangement according to claim 16, characterised in that said device (10) for transferring said enteral nutritive solution (A) and/or said fluid (B) for covering the fluid requirement comprising a tube line (11), which at one end comprises a connection piece (16) for the connection of said device for transferring said enteral nutritive solution to a connection piece of said multi-chamber bag (1, 16, 17, 22) and at the other end a connection piece (23) for the connection to a probe for said enteral nutrition.

18. The arrangement according to claim 16, characterised in that said packaging being a bag (20).

19. The multi-chamber bag according to claim 1, characterised in that said enteral nutritive solution (A) having an energy content of at least 0.8 kcal/ml.

20. The multi-chamber bag according to claim 1, characterised in that said enteral nutritive solution (A) having an energy content of at least 1 kcal/ml.
